(19) United States
(12) Patent Application Publication
(10) Pub. No.: US 2022/0186948 A1
(43) Pub. Date: Jun. 16, 2022

(54) HUMIDIFIER WITH ELECTROLYTIC STERILIZATION MODULE

(71) Applicant: JIANGMEN HENGTONG HI-TECH CO., LTD., Jiangmen, Guangdong (CN)

(72) Inventor: Hui Chen, Jiangmen (CN)

(73) Assignee: JIANGMEN HENGTONG HI-TECH CO., LTD., Jiangmen, Guangdong (CN)

(21) Appl. No.: 17/264,371

(22) PCT Filed: Jan. 20, 2021
(86) PCT No.: PCT/CN2021/072891

§ 371 Date: Jan. 29, 2021

(30) Foreign Application Priority Data
Jun. 22, 2020 (CN) 202021172463.3

(51) Int. Cl. F24F 6/12; C02F 1/461

(52) U.S. Cl. CPC F24F 6/12; F24F 2006/006; C02F 1/46104

(57) ABSTRACT

Disclosed is a humidifier with an electrolytic sterilization module, including a water storage tank and an electrolytic sterilization module arranged in the water storage tank. The electrolytic sterilization module includes a housing arranged in the water storage tank, a titanium anode mesh and a titanium cathode mesh. The titanium anode mesh and the titanium cathode mesh are arranged in the housing. The titanium anode mesh is electrically connected with a positive electrode of a power supply by a titanium anode bolt, and the titanium cathode mesh is electrically connected with a negative electrode of the power supply by a titanium cathode bolt, The titanium anode bolt and the titanium cathode bolt penetrate through the housing and the bottom of the water storage tank.

### TECHNICAL FIELD

The present disclosure relates to the field of humidifiers, and in particular, to a humidifier with an electrolytic sterilization module.

### BACKGROUND

With the development of society, people have increasingly higher requirements for quality of life. On dry days in autumn and winter, people (especially people in the north) may use humidifiers to increase the humidity of air and keep the skin moist. The existing humidifiers only have basic atomization and humidification functions in general, and bacteria in the air may be adsorbed in the mist and discharged during humidification, which is harmful to human health. Meanwhile, the bacteria are easy to breed in the water tank.

### SUMMARY

The technical problem to be solved by the present disclosure is to provide a humidifier with an electrolytic sterilization module which has simple structure and good sterilization effect.

In order to solve the above technical problem, the technical scheme of the present disclosure is as follows.

A humidifier with an electrolytic sterilization module is provided, including: a water storage tank; and an electrolytic sterilization module arranged in the water storage tank, the electrolytic sterilization module includes a housing arranged in the water storage tank and a titanium anode mesh and a titanium cathode mesh. The titanium anode mesh and the titanium cathode mesh are arranged in the housing, the titanium anode mesh is electrically connected with a positive electrode of a power supply by a titanium anode bolt, the titanium cathode mesh is electrically connected with a negative electrode of the power supply by a titanium cathode bolt, and the titanium anode bolt and the titanium cathode bolt penetrate through the housing and a bottom of the water storage tank.

In some embodiments, the housing includes an upper cover and a lower cover, the upper cover is covered on the lower cover and provided with a groove at a bottom of the upper cover, the lower cover is provided with a bump matched with the groove at a top of the lower cover, and the bump is sleeved in the groove.

In some embodiments, the upper cover is tightly connected with the lower cover by interference fit between the groove and the bump.

In some embodiments, the upper cover is tightly connected with the lower cover by ultrasonic welding.

In some embodiments, a separator made of insulating material is arranged between the titanium anode mesh and the titanium cathode mesh.

In some embodiments, the housing is provided with a first mounting hole and a second mounting hole at a top of the housing, and the titanium anode bolt and the titanium cathode bolt are respectively arranged in the first mounting hole and the second mounting hole.

In some embodiments, the titanium anode mesh and the titanium cathode mesh are respectively provided with a first through hole and a second through hole, and the titanium anode bolt and the titanium cathode bolt respectively pass through the first through hole and the second through hole contactlessly.

In some embodiments, the humidifier further includes isolation rings respectively arranged in the first through hole and the second through hole and fixedly connected with the first through hole and the second through hole, wherein the isolation rings are made of insulating material.

In some embodiments, each of the titanium anode bolt and the titanium cathode bolt is provided with a ground coil at a bottom of the each of the titanium anode bolt and the titanium cathode bolt, and the ground coils are respectively fixed to the titanium anode bolt and the titanium cathode bolt by nuts.

In some embodiments, the housing is provided with a waterproof hole at a bottom of the housing, a silicone gasket is arranged in the waterproof hole, and the titanium anode bolt and the titanium cathode bolt respectively pass through the silicone gasket.

The present disclosure has the beneficial effects that: the electrolytic sterilization module is arranged in the water storage tank, so tap water in the water storage tank is electrolyzed, Acidic electrolytic water containing hypochlorous acid is generated at the titanium anode mesh and plays a role of sterilization and disinfection after being atomized. Alkaline electrolytic water is generated at the titanium cathode mesh and has a beneficial effect on human health if absorbed by human body after being atomized. In addition, the silicone gasket can effectively prevent short circuit caused by water leakage.

### BRIEF DESCRIPTION OF THE DRAWINGS

FIG. 1 is a structural diagram of a humidifier with an electrolytic sterilization module according to an embodiment of the present disclosure;

FIG. 2 is an exploded structural diagram of a humidifier with an electrolytic sterilization module according to an embodiment of the present disclosure;

FIG. 3 is a structural diagram of a humidifier including a control device 120 according to an embodiment of the present disclosure; and

FIG. 4 is a structural diagram of a control device 120 according to an embodiment of the present disclosure.

### DETAILED DESCRIPTION

Embodiments of the present disclosure are described in detail below. Examples of the embodiments are shown in the accompanying drawings, in which the same or similar reference numerals refer to the same or similar elements or elements having the same or similar functions. The embodiments described below with reference to the accompanying drawings are examples and are merely intended to explain the present disclosure, and cannot be understood as limiting the present disclosure.

In the description of the present disclosure, it should be understood that if orientation description is involved, the orientation or position relationship indicated by, for example, “up”, “down”, “front”, “rear”, “left” and “right” is based on the orientation or position relationship shown in the drawings, and these terms are merely used to facilitate description of the present disclosure and simplify the description, but not to indicate or imply that the mentioned device or elements must have a specific orientation and must be established and operated in a specific orientation, and thus, these terms cannot be understood as a limitation to the present disclosure.

In the description of the present disclosure, “several” means one or more, and “a plurality of” means at least two. “More than”, “less than”, “exceeding, and the like” are understood as excluding this number, and “above”, “below”, “within”, and the like are understood as including this number. “First” and “second” in description are only for the purpose of distinguishing between technical features, and cannot be understood as indicating or implying relative importance or implicitly indicating the number of technical features indicated or implicitly indicating the sequence relationship of indicated technical features.

In the description of the present disclosure, unless otherwise explicitly defined, the words such as “setting”, “mounting” and “connection” should be understood in a broad sense, and those skilled in art can properly determine the specific meanings of the above words in the present disclosure with reference to the specific contents of the technical solution.

The existing humidifiers only have basic atomization and humidification functions in general, and bacteria in the air may be adsorbed in the mist and discharged during humidification, which is harmful to human health. Meanwhile, the bacteria are easy to breed in the water tank.

The existing sterilization methods include ultraviolet ray, ozone, ultrasonic wave, magnetization, static electricity and other treatment methods, which are not widely applied because of complex equipment, small flow of treated water or high treatment costs. With the development of industry, a water source is increasingly subjected to pollution by various organic substances, and it is found that a traditional chlorination disinfection method of a water supply system may produce substances harmful to human body. Therefore, the research on novel alternative sterilization methods has increasingly become a hot topic and an important development trend at home and abroad. Most of the existing commercial electric disinfection water processing devices generate chlorine dioxide, sodium hypochlorite and other disinfectants by electrolysis after adding sodium chloride and other electrolytes. After use, many substances harmful to human body are generated.

Therefore, the present disclosure provides a humidifier for sterilization based on an electrolysis method. The electrolysis method is popular as a sterilization water treatment technology with good sterilization effect, strong usability and no secondary pollution. Electrolytic sterilization may be divided into indirect sterilization and direct sterilization. Indirect sterilization means that ClO⁻, HClO₃, H₂O₂, O₂—, OH— and other substances generated by electrolysis are used to kill microorganisms. Direct sterilization means that electrolytic electrodes are used to directly act on microorganisms to make microorganisms die.

The sterilization and disinfection mechanism of the electrolysis method is as follow: 1. When water with chlorine is electrolyzed, it will generate ClO⁻ and a small amount of chlorate with higher valence, where both ClO— and chlorate are strong oxidants which have strong killing effect on microorganisms; 2. when water without chloride ion is electrolyzed, it will generate ozone, hydrogen peroxide and other strong oxidizing substances, which also have the function of sterilization; and 3. Electrolysis is directly applied on the bacterial cell bodies to destroy bacterial organisms and cause death of the bacteria.

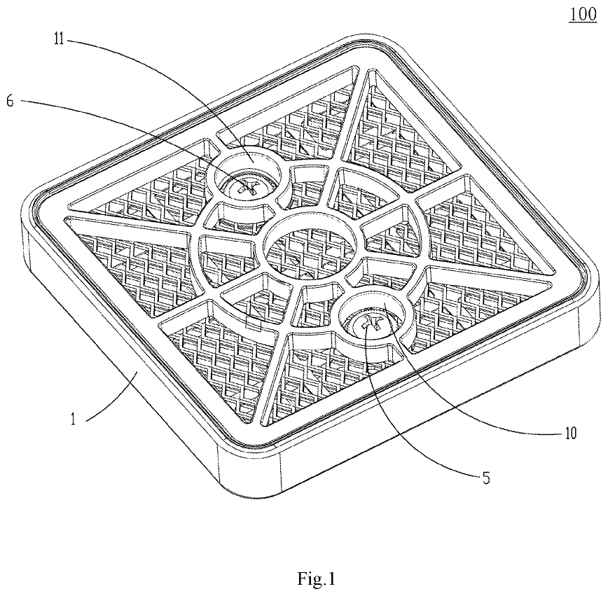

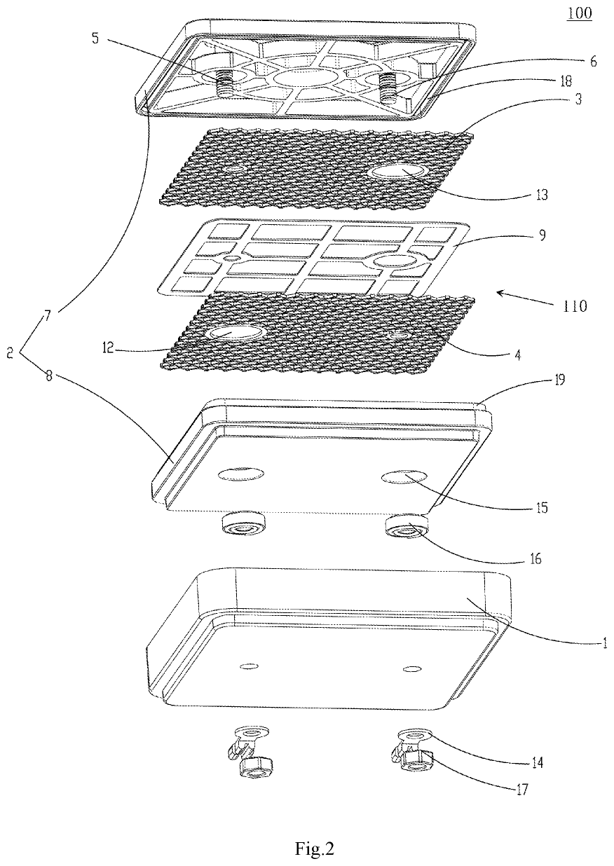

As shown in FIGS. 1-2, a humidifier 100 with an electrolytic sterilization module 110 is provided according to an embodiment of the present disclosure, including a water storage tank 1. The humidifier 100 can atomize and diffuse water in the water storage tank 1 to the outside of the humidifier 100 to humidify the environment. An electrolytic sterilization module 110 is arranged in the water storage tank 1. The electrolytic sterilization module 110 includes a housing 2 arranged in the water storage tank 1, a titanium anode mesh 3 and a titanium cathode mesh 4. The titanium anode mesh 3 and the titanium cathode mesh 4 are metal conductive meshes coated with titanium on the surface to form titanium electrodes, and have the characteristics of stable conductivity, low energy consumption and corrosion resistance. The titanium anode mesh 3 and the titanium cathode mesh 4 are arranged in the housing 2, the titanium anode mesh 3 is electrically connected with a positive electrode of a power supply by a titanium anode bolt 5, the titanium cathode mesh 4 is electrically connected with a negative electrode of the power supply by a titanium cathode bolt 6, and the titanium anode bolt 5 and the titanium cathode bolt 6 penetrate through the housing 2 and the bottom of the water storage tank 1 to be connected with the power supply. When the humidifier 100 operates, tap water is added into the water storage tank 1. Since the tap water contains chloride ions, after the electrolytic sterilization module 110 is energized to electrolyze the tap water, acidic electrolytic water containing hypochlorous acid is generated at the titanium anode mesh 3. The hypochlorous acid has strong oxidizability and sterilization effect. After sterilization, the hypochlorous acid may decompose itself, causing almost no pollution to the environment (even if raw water does not contain chloride ions, ozone, hydrogen peroxide and other strong oxidizing substances generated by electrolysis also have sterilization effect). In contrast, alkaline electrolytic water is generated at the titanium cathode mesh 4, which when atomized and inhaled by a human body, can quickly remove acidic metabolites in the human body, thus having good preventive and therapeutic effects on gastric ulcer, acid reflux, gout and muscle soreness caused by excessive acidity.

In some embodiments, the housing 2 includes an upper cover 7 and a lower cover 8. The upper cover 7 is covered on the lower cover 8 and provided with a groove 18 at the bottom. The lower cover 8 is provided with a bump 19 matched with the groove 18 at the top. Arrangement of the bump 19 within the groove 18 can realize positioning connection between the upper cover 7 and the lower cover 8.

In some embodiments, the upper cover 7 and the lower cover 8 are fixed to each other by interference fit between the groove 18 and the bump 19. Additionally or alternatively, the upper cover 7 is connected with the lower cover 8 by ultrasonic welding. Both the two exemplary fixing modes can avoid the use of bolts, thus avoiding the influence of other metal material on water electrolysis.

In some embodiments, the titanium anode mesh 3 is arranged above the titanium cathode mesh 4. A separator 9 made of insulating material is arranged between the titanium anode mesh 3 and the titanium cathode mesh 4 and is used for separating the titanium anode mesh 3 from the titanium cathode mesh 4. In some embodiments, the insulating material may be polycarbonate.

It should be understood that although FIG. 2 shows that the titanium anode mesh 3 is arranged above the titanium cathode mesh 4, the present disclosure is not limited thereto, and in the present disclosure, the titanium anode mesh 3 may also be arranged below the titanium cathode mesh 4 according to actual needs, which can also achieve the purpose of sterilization by the electrolytic sterilization module.

In some embodiments, the titanium anode mesh 3 and the titanium cathode mesh 4 are respectively provided with a first through hole 12 and a second through hole 13. The titanium anode bolt 5 and the titanium cathode bolt 6 respectively pass through the first through hole 12 and the second through hole 13 contactlessly, so as to avoid short circuit caused by the contact between the positive electrode of the power supply and the negative electrode of the power supply. Additionally or alternatively, isolation rings made of insulating material, such as silicone rings may be arranged in the first through hole 12 and the second through hole 13, respectively, so as to further avoid short circuit caused by contact between the positive electrode of the power supply and the negative electrode of the power supply.

In some embodiments, the housing 2 is provided with a first mounting hole 10 and a second mounting hole 11 at the top. The titanium anode bolt 5 and the titanium cathode bolt 6 are respectively arranged in the first mounting hole 10 and the second mounting hole 11, to facilitate positioning and mounting.

In some embodiments, the housing 2 is provided with a waterproof hole 15 at the bottom. A silicone gasket 16 is arranged in the waterproof hole 15. The titanium anode bolt 5 and the titanium cathode bolt 6 respectively pass through the silicone gasket 16. The silicone gasket 16 plays a sealing role to prevent water in the water storage tank 1 from flowing out along the titanium anode bolt 5 or the titanium cathode bolt 6 to cause short circuit. The titanium anode bolt 5 and the titanium cathode bolt 6 are provided with ground coils 14 at the bottom, and the ground coils 14 are respectively fixed to the titanium anode bolt 5 and the titanium cathode bolt 6 by nuts 17 to play a safety protection role.

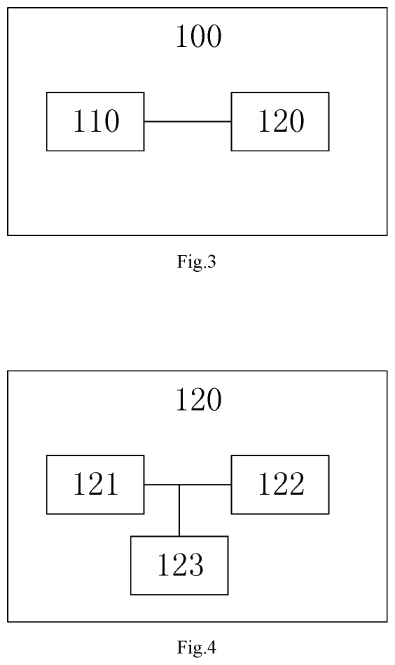

In some cases, when new water is added into the humidifier 100 or after the electrolytic sterilization module 110 runs for a period of time, the content of bacteria in the water may be low at this time. In this case, if the electrolytic sterilization module 110 is continuously energized, power is wasted. Therefore, in some embodiments, as shown in FIG. 3, the humidifier 100 may further include a control device 120 connected with the electrolytic sterilization module 110. The control device 120 is configured to detect water quality information, such as bacteria concentration in water, and determine whether to turn on the electrolytic sterilization module 110 or not according to the detected water quality information.

In some embodiments, as shown in FIG. 4, the control device 120 may include a detector 121 configured to detect the water quality information; a comparator 122 configured to compare the detected water quality information with a preset threshold; and a controller 123 configured to control the ON or OFF of the electrolytic sterilization module 110 based on a comparison result of the comparator 122. In some embodiments, the preset threshold may be set according to the specific use environment or use population. For example, the preset threshold may be set to 1000 cfu/ml, and of course, may be set to be higher or lower than 1000 cfu/ml.

In general, when water in the humidifier 100 is replaced with new humidification water, the content of bacteria in the water may be low at this time, so there is no need to turn on the electrolytic sterilization module 110. In this way, by arranging the control device 120, it is possible to determine whether the electrolytic sterilization module 110 needs to be turned on after water in the humidifier 100 is replaced with new humidification water. Specifically, when the humidifier 100 is energized, the detector 121 detects a bacteria concentration value in water and sends the bacteria concentration value to the comparator 122. The comparator 122 compares the received bacteria concentration value with a preset threshold (e.g., 1000 cfu/ml), and sends a comparison result to the controller 123. When the received bacteria concentration value is higher than the preset threshold, the controller 123 controls the electrolytic sterilization module 110 to be turned on, and the electrolytic sterilization module 110 electrolyzes tap water after being turned on, thereby achieving the purpose of electrolytic sterilization. When the received bacteria concentration value is not higher than the preset threshold, the controller 123 controls the electrolytic sterilization module 110 to be turned off, thereby achieving the purpose of saving energy and electricity.

In some embodiments, the detector 121 is configured to periodically detect water quality information, for example, every 2 hours.

In some embodiments, the detector 121 is further configured to detect water level information in the water storage tank 1, and the controller 123 is further configured to control the electrolytic sterilization module 110 to be turned off when a water level in the water storage tank 1 is lower than the titanium anode mesh 3 arranged above. When the water level in the water storage tank 1 is lower than the titanium anode mesh 3 arranged above, if the electrolytic sterilization module 110 is continuously turned on at this time, the electrolytic sterilization module 110 may not only fail to achieve the purpose of electrolytic sterilization, but may also be damaged. Therefore, this problem can be avoided by means of the above setting mode.

It should be understood that the apparatus embodiments described above are only illustrative, in which units described as separated components may be or may not be separated physically. The solution of the embodiments of the present disclosure can be achieved by selecting some or all of modules according to actual needs. Those having ordinary skill in the art can understand and implement the solution without creative efforts.

In the description of this specification, the description with reference to the terms “one embodiment”, “some embodiments”, “schematic embodiments”, “examples”, “specific examples” or “some examples” means that a particular feature, structure, material or characteristic described in connection with the embodiment or example is included in at least one embodiment or example of the utility model. In this specification, the schematic expressions of the above terms do not necessarily refer to the same embodiments or examples. Furthermore, the described specific features, structures, materials or characteristics can be combined in any suitable manner in one or more embodiments or examples.

Embodiments of the present disclosure are described in detail above in combination with the drawings. However, the present disclosure is not limited to these embodiments. For those having ordinary skill in the art, many changes, modifications, replacements and variations may be made without departing from the principles of the present disclosure, and these changes, modifications, replacements and variations should also be deemed to be fallen into the protection scope of the present disclosure.

### LIST OF REFERENCE NUMERALS

100 Humidifier
110 Electrolytic sterilization module
120 Control device
121 Detector
122 Comparer
123 Controller
1 Water storage tank
2 Housing
3 Titanium anode mesh
4 Titanium cathode mesh
5 Titanium anode bolt
6 Titanium cathode bolt
7 Upper cover
8 Lower cover
9 Separator
10 First mounting hole
11 Second mounting hole
12 First through hole
13 Second through hole
14 Ground coil
15 Waterproof hole
16 Silicone gasket
17 Nut
18 Groove
19 Bump

1. A humidifier with an electrolytic sterilization module, comprising:
a water storage tank; and
an electrolytic sterilization module arranged in the water storage tank, the electrolytic sterilization module comprising:
a housing arranged in the water storage tank; and
a titanium anode mesh and a titanium cathode mesh, wherein the titanium anode mesh and the titanium cathode mesh are arranged in the housing, the titanium anode mesh is electrically connected with a positive electrode of a power supply by a titanium anode bolt, the titanium cathode mesh is electrically connected with a negative electrode of the power supply by a titanium cathode bolt, and the titanium anode bolt and the titanium cathode bolt penetrate through the housing and a bottom of the water storage tank.

2. The humidifier with an electrolytic sterilization module of claim 1, wherein the housing comprises an upper cover and a lower cover, the upper cover is covered on the lower cover and provided with a groove at a bottom of the upper cover, the lower cover is provided with a bump matched with the groove at a top of the lower cover, and the bump is sleeved in the groove.

3. The humidifier with an electrolytic sterilization module of claim 2, wherein the upper cover is tightly connected with the lower cover by interference fit between the groove and the bump.

4. The humidifier with an electrolytic sterilization module of claim 2, wherein the upper cover is tightly connected with the lower cover by ultrasonic welding.

5. The humidifier with an electrolytic sterilization module of claim 1, wherein a separator made of insulating material is arranged between the titanium anode mesh and the titanium cathode mesh.

6. The humidifier with an electrolytic sterilization module of claim 1, wherein the housing is provided with a first mounting hole and a second mounting hole at a top of the housing, and the titanium anode bolt and the titanium cathode bolt are respectively arranged in the first mounting hole and the second mounting hole.

7. The humidifier with an electrolytic sterilization module of claim 1, wherein the titanium anode mesh and the titanium cathode mesh are respectively provided with a first through hole and a second through hole, and the titanium anode bolt and the titanium cathode bolt respectively pass through the first through hole and the second through hole contactlessly.

8. The humidifier with an electrolytic sterilization module of claim 7, further comprising: isolation rings respectively arranged in the first through hole and the second through hole and fixedly connected with the first through hole and the second through hole, wherein the isolation rings are made of insulating material.

9. The humidifier with an electrolytic sterilization module of claim 1, wherein each of the titanium anode bolt and the titanium cathode bolt is provided with a ground coil at a bottom of the each of the titanium anode bolt and the titanium cathode bolt, and the ground coils are respectively fixed to the titanium anode bolt and the titanium cathode bolt by nuts.

10. The humidifier with an electrolytic sterilization module of claim 1, wherein the housing is provided with a waterproof hole at a bottom of the housing, a silicone gasket is arranged in the waterproof hole, and the titanium anode bolt and the titanium cathode bolt respectively pass through the silicone gasket.
